(19) United States
(12) Patent Application Publication
(10) Pub. No.: US 2002/0164681 A1
(43) Pub. Date: Nov. 7, 2002

(54) Gelatins with improved gliding power, processes for their preparation and their applications

(76) Inventors: Olivier Lafargue, Isle-Sur-Sorgue (FR); Jacky David, Isle-Sur-Sorgue (FR); Georges Takerkart, Issy-Les-Moulineaux (FR)

Correspondence Address: Gary M. Nath, NATH & ASSOCIATES PLLC, 6th Floor, 1030 15th Street, N.W., Washington, DC, 20005, US

(21) Appl. No.: 10/131,525

(22) Filed: Apr. 25, 2002

Related U.S. Application Data
(63) Continuation-in-part of application No. 09/858,894, filed on May 17, 2001.

(30) Foreign Application Priority Data
May 2, 2001 (FR) 0105862

(51) Int. Cl. C12P 21/06; G03C 1/005; G03C 1/494; C07K 14/78

(52) U.S. Cl. 435/68.1; 530/354; 430/642

(57) ABSTRACT

The invention relates to gelatins with improved gliding power, to processes for their preparation and their applications in particular in the manufacture of capsules.

The said gelatins contain anionic or nonionic surfactant compounds.

The present invention relates to gelatins with improved gliding power, to processes for their preparation and their applications in particular in the manufacture of capsules.

The preparation of gelatin capsules from gelatin was described by the French A. B. MOTHES in patent No. 9690 filed in 1834. The process then became widespread and numerous patents have been filed.

In the years which followed, processes for the preparation of gelatin capsules from gelatin were constantly improved in order to satisfy increasingly demanding technical specifications.

Gelatin is the principal constituent of the capsule. It includes gelatins of high purity prepared from collagenic raw materials present in the skin and the bones of animals. There are two types of gelatin: that of type A, having an isoelectric point of between 7.5 and 9, extracted by treating with a hot aqueous solution of acid, generally H₂SO₄, pig skins, bovine skins or bones which have been crushed, freed of dust and demineralized; and that of type B, with an isoelectric point of close to 5 which is obtained by treating demineralized bones or bovine skins with a basic aqueous solution, lime or sodium hydroxide in general.

The hard capsule is composed of two cylindrical parts, namely a body with a hemispherical base and a cap having the same shape but shorter.

Before filling, the bodies of the capsules pass through a constricted passage and become inserted into a rotating disk to allow filling. At this stage, it is important to avoid slowing down in the constricted passages and interactions of the adherence type or electrostatic phenomena for the bodies of the capsules with the powder.

The inner diameter of the cap being practically equal to the outer diameter of the body, when the two parts are assembled, a reservoir with a very reproducible volume is obtained. Given that this involves a tight adjustment and that the assembling is carried out on machines with a high throughput, it is crucial that the surfaces which have to overlap should glide perfectly relative to each other. If that is not the case, the two parts of the capsule are rejected, which reduces the output of the machine and increases the rejection rate.

The problem to be solved therefore consists in improving the gliding property of the walls of the capsule relative to the materials in the machines (constricted passages), the powders and the other capsules.

The gliding property of the walls of the capsule may be evaluated by preparing films of the gelatin used.

The test consists in measuring the inclination from which two films of the same gelatin glide against each other. More elaborate techniques may also be used to characterize the surface condition such as the so-called AFM “atomic force microscopy” technique as described for example in S. Magonov et al., Surface Analysis with STM and AFM, VCH Ed., 1996.

The gliding test developed by the inventors is the following:

30 g of gelatin are weighed in a bottle and then 70 g of demineralized water are added. After homogenization, the solution is allowed to stand (for swelling) for 30 min. The bottles are then placed in a bath at 65° C. After 2 h, the solution is stirred and then incubated for 19 h on a water bath at 50° C.

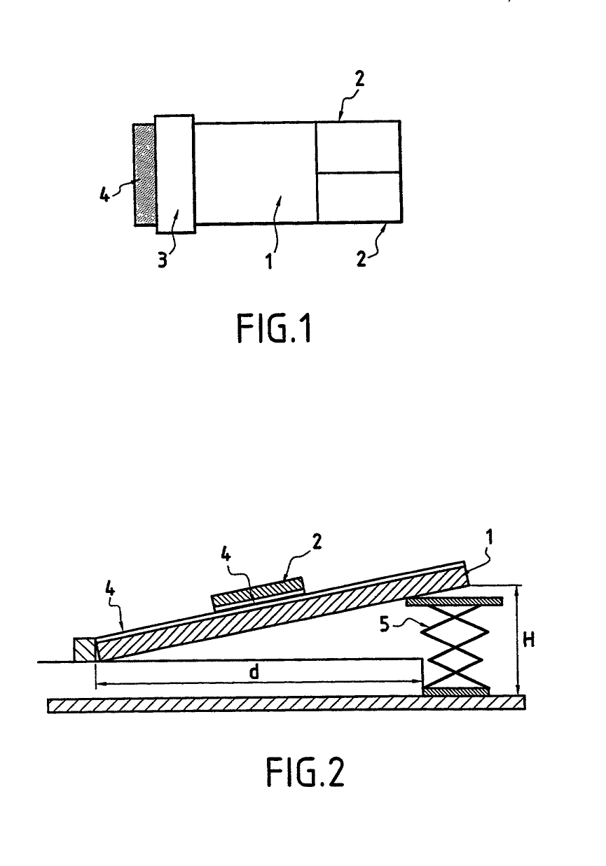

Three gelatin films are then prepared with the aid of a film applicator represented in FIG. 1.

The latter is a top view showing:

in (1), a 200 mm×200 mm glass plate,

in (2), two 100 mm×50 mm glass plates,

in (3), a reel whose slit is set at 0.8 mm, and

in (4), the gelatin film.

The gelatin solution is poured into the reel which moves at the speed of 5 cm/s.

The 3 plates, once separated, are placed in an incubator at 27° C., in an atmosphere with 40% relative humidity for 18 h.

To measure the gliding properties, the system represented in FIG. 2 is used.

The plate (1) coated with gelatin film (4) is placed so as to create an inclined plane, the gelatin film being on the top surface, and the plate (2), also coated with a gelatin film, is caused to glide over it according to an inclination set by an elevating system (5). The distance d is 130 mm.

The height is then gradually reduced until the small plate no longer glides spontaneously. A value Hₘₘ, characteristic of the gliding properties, is then determined, the gliding properties being better the lower the Hₘₘ value.

It has been possible to demonstrate by this test that some gelatins had remarkable gliding properties, whereas others did not glide at all, and it has been possible to establish a classification scale.

It has thus been found that the gliding property could be linked to the presence of certain compounds in gelatin. It has been possible to determine their nature: they are natural anionic surfactant compounds derived from the raw material used, but which are only present in the gelatin in a very small quantity. Surprisingly, it has been found that it is possible to significantly increase the content of anionic surfactant compounds in the gelatin compared with the natural content. A method of assay has been developed. It consists in complexing them with a quaternary ammonium (cetyldimethylbenzylammonium chloride type) and in assaying the excess ammonium with bromophenol blue in UV at 605 nm.

The invention therefore relates, according to a first aspect, to a gelatin with improved gliding power containing an increased content of natural anionic surfactant compounds, which compounds improve the surface condition of the films prepared from this gelatin.

The content of anionic surfactant compounds in a standard gelatin is generally lower than about 50 ppm.

Advantageously, the quantity of anionic surfactant compounds of the gelatin with improved gliding power according to the invention is between 200 and 10 000 ppm, preferably between 300 and 4 000 ppm.

The said anionic surfactant compounds are in particular derivatives of fatty acids such as for example of stearic, palmitic, oleic, myristic or pentadecanoic acids.

Surprisingly, it has also been found that these anionic surfactant compounds are released during certain stages of the manufacture in the gelatins and that their content could be increased by certain treatments using proteolytic enzymes. Gelatins according to the invention are thus obtained which have remarkable gliding properties and which lead to increased performances during the manufacture of capsules.

The invention therefore relates, according to a subsequent aspect, to a process for preparing a gelatin as defined above, in which the anionic surfactant compounds are released in situ by subjecting a collagenic raw material to an enzymatic treatment during the hot extraction of the gelatin.

The enzymatic treatment consists in adding to the extraction vessel a small quantity of at least one proteolytic enzyme and in controlling the temperature and the duration of extraction so as to limit the degradation of the gelatin and to preserve in particular the desired viscosity.

By way of examples of proteases, there may be mentioned without limitation neutral, alkaline or acid proteases, or an enzymatic composition containing them, like those commercially available, such as for example neutrase® provided by NOVO which comprises mainly a Bacillus subtilis metalloprotease, alcalase® provided by NOVO containing subtilisin A, or pronase or papain.

Preferably, the pH during this addition is from 5 to 8, in particular 7, and the temperature is equal to or greater than about 50° C., preferably from 60 to 80° C.

The duration of the extraction will be preferably from 2 to 6 h.

According to an aspect of the invention, the enzymatic treatment is carried out at a late step of the extraction, while one or several extraction steps are beforehand carried out without the presence of enzyme.

Advantageously, said enzymatic treatment is carried out after extraction of 30 to 40% by weight of the total gelatin.

For the purposes of the invention, the gelatin is extracted from collagenic raw materials by conventional processes as described for example in “The Science and Technology of Gelatin” A. G. Ward and A. Courts, Academic Press Ed., 1977.

Preferably, said collagenic raw material is not a raw material as such, but has been, before extraction, submitted to a preparation by an acid or alkaline treatment.

By “preparation by an acid treatment” or “preparation by an alkaline treatment”, it is understood the treatments which are performed prior to extraction, as described in usual processes, such as for instance soaking the raw material for more or less time in a bath of an organic or mineral acid, or in an alkaline solution such as a sodium or potassium hydroxide solution.

It can be again referred in this respect to the above-cited book “The Science and Technology and Gelatin”.

Collagenic raw materials which can be advantageously used are for example ossein (demineralized bone), bovine skin, pig skin or fish skin, limed ossein being preferred.

According to a preferred aspect, the gelatin is extracted from limed ossein and the enzymatic treatment is carried out in situ during extraction, using an amount of 0.2 g to 5 g of enzyme(s)/tonne of limed ossein.

The invention also relates, according to one of its aspects, to the use of the gelatin with improved gliding power, as defined above, for the manufacture of films and capsules.

According to another of its aspects, the invention further relates to a process for preparing the gelatin as defined above, consisting in adding anionic surfactant compounds which are soluble in an aqueous medium to a solution of gelatin, for example at the end of the extraction.

Using anionic surfactant compounds which are soluble in the presence of gelatin is particularly advantageous in that the clarity of the gelatin solution is maintained.

Preferably, the said anionic surfactant compounds are added in an amount of 200 to 10 000 ppm, preferably 300 to 4 000 ppm.

Preferably, the said anionic surfactant compounds are chosen from fatty acid salts such as sodium or potassium stearate, palmitate, oleate, myristate or pentadecanoate, the sodium salts being preferred, or phospholipids such as lecithins.

According to a subsequent aspect, the invention also relates to a gelatin with improved gliding power containing nonionic surfactant compounds.

Indeed, it has also been found that the addition of nonionic surfactant compounds to a solution of gelatin after extraction made it possible to improve the gliding power and to obtain a gelatin for which the Hₘₘ value of the films produced with this gelatin was significantly reduced relative to the gelatin not supplemented with nonionic surfactant compounds.

Preferably, 200 to 10 000 ppm, in particular from 300 to 4 000 ppm of the said nonionic surfactant compounds are added to the solution of gelatin.

The subject of the invention is therefore also a process for obtaining such a gelatin as described above, as well as the use of this gelatin for the manufacture of films and capsules.

Preferably, the nonionic surfactant compounds are chosen from sorbitan derivatives such as for example sorbitan monostearate (Span® 60) or sorbitan monooleate (Span® 80).

The invention is illustrated by the examples below:

### EXAMPLE 1

15 tonnes of limed ossein, obtained according to a conventional process, are loaded with water into an extraction vessel. 10 g of neutral metalloprotease from B. subtilis (B500 from GIST-BROCADES) are added to the vessel as soon as the temperature rises to 75° C., the pH being maintained at 6.5. After extracting the gelatin for 3 to 4 h, the solution is pasteurized so as to inactivate the enzyme, filtered, demineralized and then gelled and dried in a conventional manner in a ventilated dryer with controlled temperature.

A gelatin having a content of anionic surfactant compounds of 827 ppm and an Hₘₘ value=25 mm is obtained, which corresponds to a satisfactory gliding test.

### EXAMPLE 2

400 ppm of sodium palmitate are added to a solution containing 30% of pig skin gelatin having no gliding properties.

A gelatin is thus obtained which has a content of anionic surfactant compounds of 700 ppm and a Hₘₘ value=30 mm, whereas it was 70 mm before addition.

### EXAMPLE 3

1 000 ppm of polysorbate 80 (SIGMA) are added to a solution containing 30% of gelatin extracted from limed ossein having no gliding properties. A gelatin is thus obtained which has an Hₘₘ value=30 mm, whereas it was 70 mm before addition.

What is claimed is:

1. Gelatin with improved gliding power, characterized in that it contains an increased content of natural anionic surfactant compounds which improve the surface condition of the films prepared from the said gelatin.

2. A gelatin according to claim 1, characterized in that the content of anionic surfactant compounds is between 200 and 10 000 ppm, preferably between 300 and 4 000 ppm.

3. A process for preparing a gelatin according to either of claims 1 and 2, wherein the anionic surfactant compounds are released in situ by subjecting a collagenic raw material to an enzymatic treatment during hot extraction of the gelatin.

4. The process for preparing a gelatin accotrding to any one of claims 1 to 3, wherein said collagenic raw material has, prior to extraction, been submitted to a preparation by an acid treatment or an alkaline treatment.

5. The process according to claim 4, wherein the gelatin is extracted from a collagenic raw material such as ossein, bovine skin, pig skin or fish skin.

6. The process according to claim 3, 4 or 5, wherein the enzyme(s) used for the enzymatic treatment is(are) chosen from neutral, alkaline or acid proteases such as metalloproteases from B. subtilis, subtilisin A, pronase, papain and compositions containing them.

7. The process according to any one of claims 3 to 6, wherein the said enzyme(s) is(are) added to the extraction vessel at a pH of 5 to 8 and at a temperature equal to or greater than 50° C.

8. The process according to any one of claims 3 to 7, wherein the enzymatic treatment is carried out at a late step of the extraction, while one or several extraction steps are beforehand carried out without the presence of enzyme.

9. The process according to claim 8, wherein said enzymatic treatment is carried out after extraction of 30 to 40% by weight of the total gelatin.

10. The process according to any one of claims 3 to 9, wherein the gelatin is extracted from limed ossein and the enzymatic treatment is carried out in situ during the extraction using an amount of 0.2 g to 5 g of enzyme(s)/tonne of limed ossein.

11. The process for preparing a gelatin according to either of claims 1 and 2, wherein anionic surfactant compounds which are soluble in an aqueous medium are added to a solution of gelatin after extraction.

12. The process according to claim 11, wherein the said anionic surfactant compounds are added in an amount of 200 to 10 000 ppm, preferably 300 to 4 000 ppm.

13. The process according to claim 11 or 12, wherein the said anionic surfactant compounds are chosen from fatty acid salts and phospholipids.

14. Gelatin with improved gliding power, characterized in that it contains nonionic surfactant compounds which improve the surface condition of the films prepared from the said gelatin.

15. The gelatin according to claim 14, characterized in that the content of nonionic surfactant compounds is between 200 and 10 000 ppm, preferably between 300 and 4 000 ppm.

16. The process for preparing a gelatin according to claim 14 or 15, wherein nonionic surfactant compounds are added to a solution of gelatin after the extraction, in order to improve the gliding power of said gelatin.

17. The process according to claim 16, wherein the said nonionic surfactant compounds are added in an amount of 200 to 10 000 ppm, preferably 300 to 4 000 ppm.

18. Process according to claim 16 or 17, wherein the said nonionic surfactant compounds are chosen from sorbitan derivatives such as sorbitan monostearate and sorbitan monooleate.

19. Use of a gelatin according to one of claims 1, 2, 14 or 15, for the manufacture of films.

20. Use of a gelatin according to one of claims 1, 2, 14 or 15, for the manufacture of capsules.
